(19) United States
(12) Patent Application Publication
(10) Pub. No.: US 2024/0387059 A1
(43) Pub. Date: Nov. 21, 2024

(54) CAREGIVER GUIDANCE SYSTEM

(71) Applicant: Hill-Rom Services, Inc., Batesville, IN (US)

(72) Inventors: Autumn S. Thompson, Philadelphia, PA (US); Stacie L. Brough, Syracuse, NY (US); Anandi Mahadevan, Murrysville, PA (US); Kathryn Piston, Syracuse, NY (US)

(21) Appl. No.: 18/650,473

(22) Filed: Apr. 30, 2024

Related U.S. Application Data
(60) Provisional application No. 63/502,196, filed on May 15, 2023.

(51) Int. Cl. G16H 70/20; A61B 5/00; G16H 40/20

(52) U.S. Cl. CPC G16H 70/20; A61B 5/7282; A61B 5/742; G16H 40/20

(57) ABSTRACT

A caregiver guidance system for generating at least one clinical recommendation is disclosed. The system receives caregiver actions data associated with an experienced caregiver and patient data associated with a patient. The system analyzes the caregiver actions data to identify at least one action performed by the experienced caregiver in response to a patient event detected from the patient data. The system correlates the at least one action performed by the experienced caregiver to the patient event. The system determines a clinical recommendation based on the correlation between the at least one action and the patient event. The system determines when the patient event occurs while an inexperienced caregiver is managing the patient event. The system presents the clinical recommendation to the inexperienced caregiver.

### BACKGROUND

When a caregiver leaves their employment at a healthcare facility, the caregiver is typically replaced with a new caregiver. In some instances, the new caregiver may not have the same nursing experience or familiarity with the healthcare facility or a specific clinical care environment within the healthcare facility such that training the new caregiver is typically necessary. However, training new caregivers typically involves significant resources.

### SUMMARY

In general terms, the present disclosure relates to a system for providing clinical decision support to caregivers. In one possible configuration, clinical decision support guidance is displayed for new or inexperienced caregivers to guide clinical workflows. Various aspects are described in this disclosure, which include, but are not limited to, the following aspects.

One aspect relates to a caregiver guidance system for generating at least one clinical recommendation, the system comprising: at least one processing device; and a memory device storing instructions which, when executed by the at least one processing device, cause the at least one processing device to: receive caregiver actions data associated with an experienced caregiver and patient data associated with a patient; analyze the caregiver actions data to identify at least one action performed by the experienced caregiver in response to a patient event detected from the patient data; correlate the at least one action performed by the experienced caregiver to the patient event; determine a clinical recommendation based on the correlation between the at least one action and the patient event; determine when the patient event occurs while an inexperienced caregiver is managing the patient event; and present the clinical recommendation to the inexperienced caregiver.

Another aspect relates to a method for determining a clinical recommendation comprising: receiving caregiver actions data associated with an experienced caregiver and patient data associated with a patient; analyzing the caregiver actions data to determine at least one action performed by an experienced caregiver in response to a patient event detected from the patient data; correlating the at least one action to the patient event; determining a clinical recommendation based on the correlation between the at least one action and the patient event; determining when the patient event occurs when an inexperienced caregiver is managing the patient event; and presenting the clinical recommendation to the inexperienced caregiver.

Another aspect relates to a communications device for displaying at least one clinical recommendation, the communications device comprising: at least one processing device; and a memory device storing instructions which, when executed by the at least one processing device, cause the at least one processing device to: detect a patient event; determine a level of experience of a caregiver proximate the patient event; receive at least one clinical recommendation when the level of experience of the caregiver is below a predetermined threshold; present the at least one clinical recommendation to the caregiver.

### DESCRIPTION OF THE FIGURES

The following drawing figures, which form a part of this application, are illustrative of the described technology and are not meant to limit the scope of the disclosure in any manner.

FIG. 1 illustrates an example of a clinical care environment that includes a caregiver guidance system for generating clinical decision support guidance.

FIG. 2 schematically illustrates an example of the caregiver guidance system of FIG. 1.

FIG. 3 schematically illustrates an example of a communications device that can be used by a caregiver in the clinical care environment of FIG. 1.

FIG. 4 schematically illustrates another example of the caregiver guidance system of FIG. 1.

FIG. 5 schematically illustrates an example of a method of generating clinical decision support guidance that can be performed by the caregiver guidance system of FIG. 1.

FIG. 6 schematically illustrates an example of caregiver actions data and the patient data used by the caregiver guidance system of FIG. 1 to generate guidance system outputs.

FIG. 7 schematically illustrates examples of data analysis that can be performed in the method of FIG. 5 for generating the guidance system outputs.

FIG. 8 schematically illustrates an example of the guidance system outputs that can be generated by the caregiver guidance system of FIG. 1.

FIG. 9 illustrates an example of the guidance system outputs that can be generated by the caregiver guidance system of FIG. 1.

FIG. 10 illustrates another example of the clinical decision support guidance that can be generated by the caregiver guidance system of FIG. 1.

FIG. 11 illustrates an example of a communications device displaying an example of clinical decision support guidance generated by the caregiver guidance system of FIG. 1.

FIG. 12 illustrates an example of a communications device displaying another example of clinical decision support guidance generated by the caregiver guidance system of FIG. 1.

FIG. 13 schematically illustrates an example of an alert received when a confidence rating produced by the caregiver guidance system of FIG. 1 is below a predetermined threshold.

FIG. 14 schematically illustrates an example of an alert received when a recommendation produced by the caregiver guidance system of FIG. 1 is escalated for further review by an experienced caregiver.

FIG. 15 schematically illustrates an example of an alert received when an inexperienced caregiver does not follow a clinical recommendation produced by the caregiver guidance system of FIG. 1.

FIG. 16 schematically illustrates an example of a method of determining a clinical recommendation that can be performed by the caregiver guidance system of FIG. 1.

### DETAILED DESCRIPTION

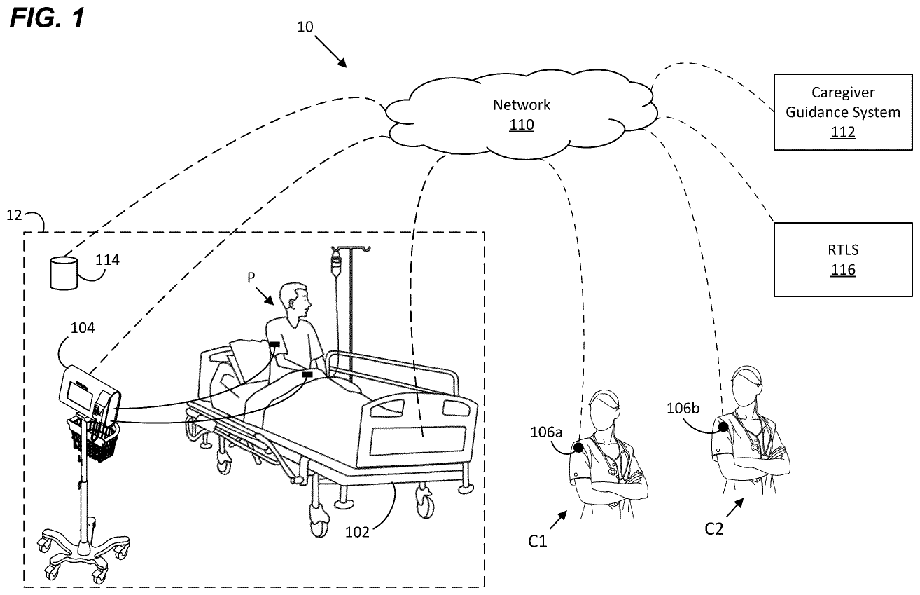

FIG. 1 illustrates an example of a clinical care environment 10 that includes a caregiver guidance system 112 for generating clinical decision support guidance. The clinical care environment 10 can be located within a healthcare facility such as a hospital, a nursing home, a rehabilitation center, a long-term care facility, and the like.

In the example shown in FIG. 1, a patient P is located in a patient environment 12, which can include a room or other designated area within the clinical care environment 10. For example, the patient environment 12 can include a patient room, a department (e.g., emergency department), a clinic, a ward, or other area within the clinical care environment 10. In this example, caregivers C are shown as being located outside of the patient environment 12.

As shown in FIG. 1, the patient P is supported on a patient support apparatus 102 inside the patient environment 12. In some examples, the patient support apparatus 102 is a hospital bed, or similar type of apparatus. In some instances, the patient support apparatus 102 includes aspects of the patient support apparatuses described in U.S. Pat. No. 8,717,181 B2, entitled BED EXIT ALERT SILENCE WITH AUTOMATIC RE-ENABLE, issued on May 6, 2014, and U.S. Pat. No. 11,172,892 B2, entitled PATIENT SUPPORT APPARATUS HAVING VITAL SIGNS MONITORING AND ALERTING, issued on Nov. 16, 2021, the disclosures of which are hereby incorporated by reference in their entireties.

The patient support apparatus 102 includes one or more sensors that measure one or more physiological parameters of the patient P. The one or more physiological parameters are recorded over time as patient data 410. For example, the one or more sensors can measure one or more vital signs such as heart rate and respiration rate. In further examples, the one or more sensors can measure patient weight, patient motion, and patient activity level. In further examples, the one or more sensors can detect patient exit and fall risk.

As further shown in FIG. 1, the patient P is connected to a monitoring device 104 positioned next to the patient support apparatus 102 inside the patient environment 12. The monitoring device 104 includes one or more sensors that can measure physiological parameters of the patient such as blood oxygen saturation, body temperature, blood pressure, pulse/heart rate, respiration rate, electrocardiogram (EKG), and the like. In some examples, the monitoring device 104 is a spot monitor that continuously monitors a health status of the patient P. The monitoring device 104 records measurements from the one or more sensors as patient data 410.

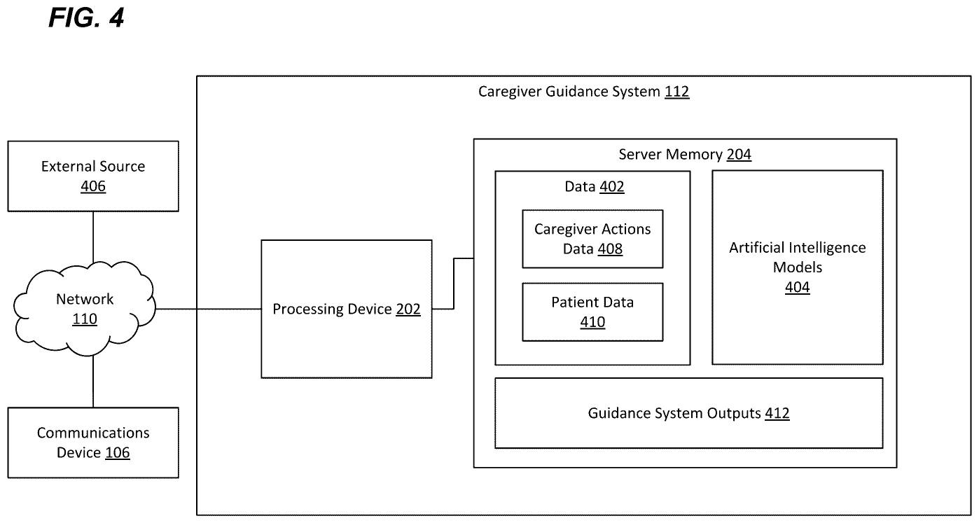

Furthermore, in some examples, the monitoring device 104 records caregiver actions data 408 (see FIG. 4) as a caregiver C performs a clinical intervention to improve the patient's condition. As an illustrative example, the caregiver actions data 408 can include the caregiver C choosing to elevate the patient P's head to reduce the patient P's risk of accidentally taking fluids and particles into the patient's lungs when the patient P is eating or drinking.

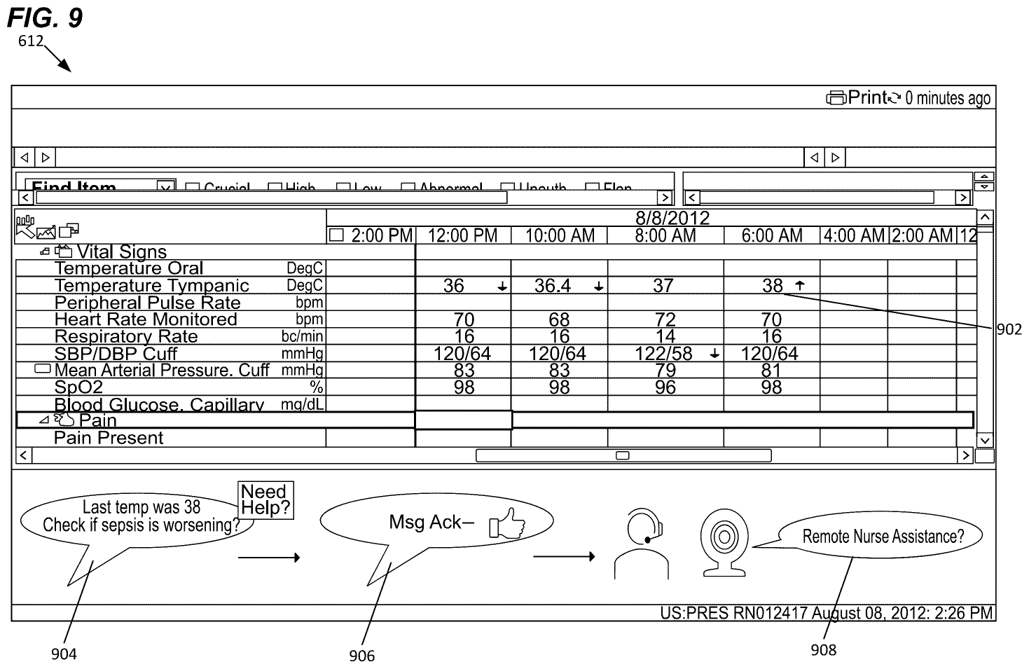

The monitoring device 104 can include an alarm system that generates alarms based on data obtained from the one or more sensors included on the monitoring device 104. As an illustrative example, when an individual physiological parameter (e.g., temperature, or blood oxygen saturation, as illustrated and described in further detail with respect to FIGS. 9-14) falls outside of a healthy range, the alarm system of the monitoring device 104 triggers an alarm. The alarms generated on or by the monitoring device 104 can be transmitted from the patient environment 12 to the caregiver guidance system 112 via the network 110. In another example, patient data 410 and caregiver actions data 408 recorded by the monitoring device 104 can be transmitted to the caregiver guidance system 112 via the network 110. The caregiver guidance system 112 can analyze the patient data 410 and caregiver actions data 408 to decide whether to issue at least one alerts and/or recommendation to a caregiver C.

The patient environment 12 further includes a fixed reference point 114 that can function as a beacon for detecting wireless signals from the patient P and/or the caregivers C inside the patient environment 12. The fixed reference point 114 transmits the wireless signals detected from the patient P and/or the caregivers C to a real-time locating system (RTLS) 116 via the network 110. The RTLS 116 uses the wireless signals received from the fixed reference point 114 to determine whether the patient P and the caregivers C are located in the patient environment 12. While a single fixed reference point is shown in FIG. 1, fixed reference points 114 can be distributed throughout the clinical care environment 10 allowing the RTLS 116 to track the locations of the patient P and the caregivers C in the clinical care environment 10. The RTLS 116 can transmit data obtained by tracking the locations of the patient P and the caregivers C to the caregiver guidance system 112 over the network 110.

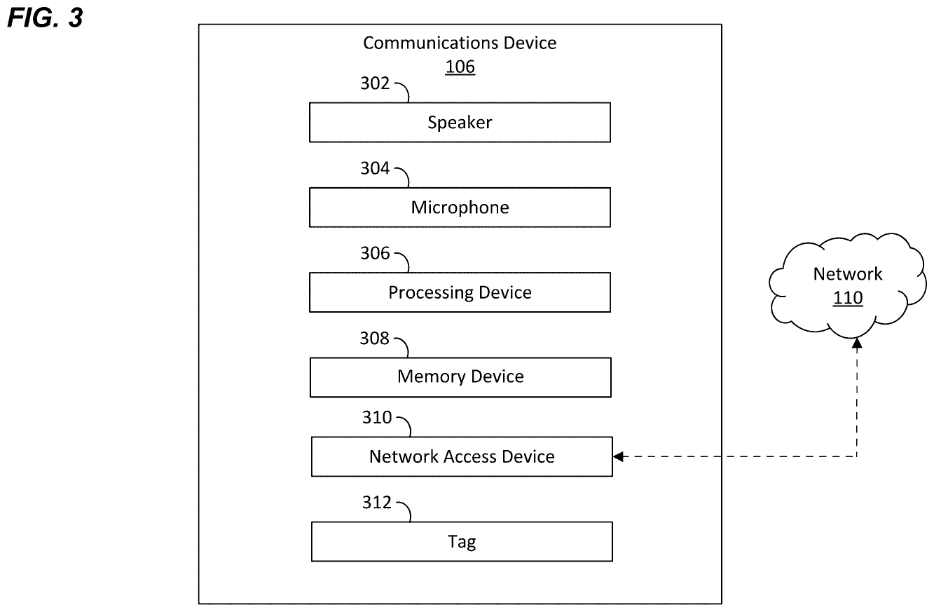

As an illustrative example, the fixed reference point 114 can detect wireless signals from tags worn by the patient P and/or by the caregivers C such as radio frequency (RF) signals, optical (e.g., infrared) signals, or acoustic (e.g., ultrasound) signals. In a further illustrative example, the fixed reference point 114 can detect Bluetooth signals emitted by devices carried by the caregivers C such as communications devices 106. In some instances, the tags that are detectable by the fixed reference point 114 are attached to the communications devices 106 (see tag 312 shown in FIG. 3, which will be described in more detail below). The communications devices 106 include any electronic device capable of transmitting and receiving communications. In some examples, the communications devices 106 include smartphones, portable tablet computers, the monitoring device 104, and other devices.

The RTLS 116 uses the wireless signals detected by the fixed reference point 114 to determine whether a caregiver C has entered the patient environment 12, or whether the patient P has absconded from the patient environment 12. Furthermore, an alarm and/or recommendation may be issued in instances when a patient event is occurring that requires immediate attention from a caregiver C and the caregiver C is not present within the patient environment 12. In certain examples, the alarm and/or recommendation may be issued in instances when the patient P absconds from the patient environment 12 without authorization. In certain examples, it is desirable to record patient movements and nurse actions using the RTLS 116 or other camera-based system to capture actions that otherwise would not be documented by a caregiver C.

Additional types of devices and systems can be positioned inside the patient environment 12 to monitor a status of the patient P. These additional types of devices can include or otherwise be connected to alarm systems that trigger additional types of alarms indicating a need for immediate intervention by the caregivers C. These additional types of devices can include video cameras that monitor the patient P inside the patient environment 12.

The patient support apparatus 102, the monitoring device 104, and the fixed reference point 114 are each connected to the network 110. When an alarm is triggered by data collected from any one of these devices inside the patient environment 12, the alarm can be communicated to the caregiver guidance system 112 via the network 110. When an alarm is triggered, alerts and recommendations can be created by the caregiver guidance system 112 and send sent to the caregiver guidance system 112 via the network 110.

The caregiver guidance system 112 routes alerts and notifications based on the alarms triggered inside the patient environment 12 to communications devices 106 worn by the caregivers C. Additionally, the caregiver guidance system 112 can open a communications channel on the communications devices 106 allowing the caregivers C to instantly communicate with one another while an alarm condition is ongoing in the patient environment 12.

In the example illustrated in FIG. 1, the clinical care environment 10 includes at least first caregiver C1, and a second caregiver C2, wherein the first caregiver C1 illustrates an inexperienced nurse and the second caregiver C2 is illustrates an experienced nurse. The first and second caregivers C1, C2 that are illustrated in FIG. 1 are provided by way of illustrative example, and it is contemplated that the number of caregivers C in the clinical care environment 10 can be significantly greater than the two caregivers shown in FIG. 1.

Each caregiver C wears or otherwise carries a communications device 106. For example, the communications devices 106 can be clipped or pinned onto the clothing worn by the caregivers or can be attached to a lanyard or other type of accessory worn by the caregivers. The communications devices 106 allow hands-free voice communications between the caregivers. Also, the communications devices 106 receive secure text messages and alert notifications, allow the caregivers C to broadcast voice and text messages to groups of caregivers in the clinical care environment 10, initiate and join conference calls, and schedule reminders. In some examples, the communications devices 106 can include smartphones, tablet computers, and other similar types of portable computing devices equipped with telecommunications. The caregiver guidance system 112 manages the data and voice communications, which are communicated over the network 110, between the communications devices 106 worn by the caregivers. In some examples, the alert notifications and recommendations are generated from the caregiver guidance system 112 and sent to the caregivers C via the network 110.

The network 110 facilitates data communication between the devices inside the patient environment 12, including the patient support apparatus 102, the monitoring device 104, the fixed reference point 114, and between the caregiver guidance system 112, the RTLS 116, and the caregiver guidance system 112, which can be located outside of the patient environment 12. In some examples, the network 110 can also facilitate data communication between the communications devices 106a, 106b.

The network 110 can include various devices and connections between the devices to enable data communication among the devices. The network 110 can include routers and other networking devices. The network 110 can include any type of wired or wireless connection, or any combinations thereof. Examples of wireless connections can include cellular network connections such as 4G or 5G. Wireless connections in the network 110 can further include Bluetooth, Wi-Fi, and other similar types of wireless technologies.

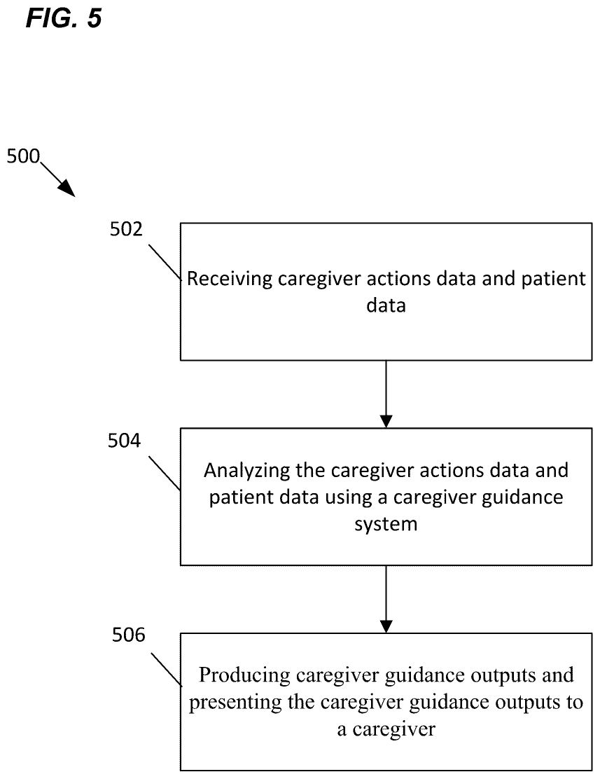

The caregiver guidance system 112 processes data received over the network 110 from the patient support apparatus 102, monitoring device 104, the communications devices 106, the caregiver guidance system 112, the fixed reference point 114, and the RTLS 116. An exemplary architecture of the caregiver guidance system 112 is described and illustrated in further detail with respect to FIG. 4. Processes performed by the caregiver guidance system 112 are described and illustrated in further detail with respect to FIGS. 5-15.

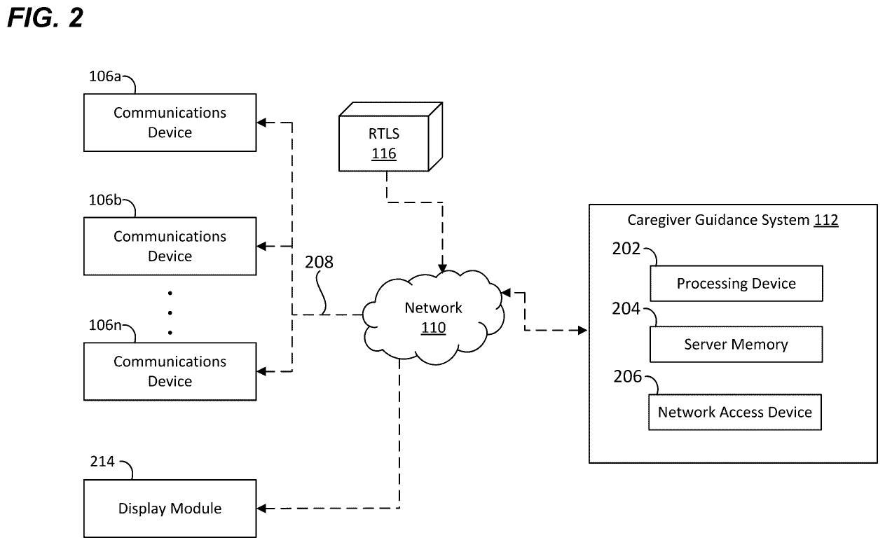

FIG. 2 schematically illustrates an example of the caregiver guidance system 112. In this example, the caregiver guidance system 112 is in communication over the network 110 with the communications devices 106 worn by the caregivers C. In this illustrative example, a first communications device 106a is worn by a first caregiver C1 who is an inexperienced nurse, a second communications device 106b is worn by a second caregiver C2 who is an experienced nurse, and any number of communications devices 106 can be used by a plurality of caregivers to communicate the caregiver actions data 408 over the network 110.

The caregiver guidance system 112 includes a processing device 202. The processing device 202 is an example of a processing unit such as a central processing unit (CPU). The processing device 202 can include one or more central processing units (CPU). In further examples, the processing device 202 includes digital signal processors, field-programmable gate arrays, and similar electronic computing circuits.

The caregiver guidance system 112 includes a server memory 204 that stores data and instructions for execution by the processing device 202. The server memory 204 includes computer readable media, including any media accessible by the caregiver guidance system 112. For example, computer readable media includes computer readable storage media and computer readable communication media.

Computer readable storage media includes volatile and nonvolatile, removable, and non-removable media implemented in any device configured to store information such as computer readable instructions, data structures, program modules, or other data. Computer readable storage media can include random access memory, read only memory, electrically erasable programmable read only memory, flash memory, and other memory technology, including any medium that can be used to store information that can be accessed by the caregiver guidance system 112. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The caregiver guidance system 112 further includes a network access device 206. The network access device 206 operates to communicate with other computing devices over the network 110 such as the communications devices 106 worn by the caregivers C. Examples of the network access device 206 include wired network interfaces and wireless network interfaces.

In some examples, the caregiver guidance system 112 facilitates communications between the communications devices 106a-106n worn by the caregivers C via the network 110. In some examples, communications are sent between the communications devices 106a-106n when an alarm condition is detected in the patient environment 12, such as when a patient event is likely to occur or when the patient event is occurring.

The caregiver guidance system 112 receives and processes data from the communications devices 106, the RTLS 116, and other systems as shown in FIG. 1. The caregiver guidance system 112 includes a processing device 202, and a server memory 204. Processed information can be transmitted to various components of the clinical care environment 10 via the network 110. The caregiver guidance system 112 is illustrated and described in further reference with respect to FIG. 4. Examples of processed information transmitted over the network 110 is described and illustrated with respect to FIGS. 5-15.

The communications device 106 displays processed information transmitted over the network 110. In some examples the communications device 106 displays processed information, such as clinical decision recommendations, received over the network 110 from the caregiver guidance system 112. In some examples, the communications device 106 includes a caregiver call system, a patient dashboard, a nurse station, or a mobile application.

FIG. 3 schematically illustrates an example of a communications device 106 that can be used by a caregiver in the clinical care environment 10. As shown in FIG. 3, the communications device 106 includes a speaker 302 that converts audio signals transmitted over the network 110 into sound. The audio signals can include audio signals received from the communications devices 106 worn by the other caregivers in the clinical care environment 10.

The communications device 106 also includes a microphone 304 that converts voice communications from the caregiver into electrical signals that can be transmitted over the network 110 to the communications devices 106 worn by the other caregivers in the clinical care environment 10. The communications device 106 can further include a tag 312 that can be detected by the fixed reference point 114 inside the patient environment 12 (see FIG. 1).

The communications device 106 further includes at least one processing device 306, a memory device 308, and a network access device 310, that can be similar to the processing device 202, the server memory 204, and the network access device 216 of the caregiver guidance system 112, described above.

FIG. 4 schematically illustrates another example of the caregiver guidance system 112. The caregiver guidance system 112 receives data 402 from an external source 406 over the network 110 and processes the data 402. Furthermore, the caregiver guidance system 112 stores the data 402 within the server memory 204. The external source 406 includes any source that transmits the data 402 to the caregiver guidance system 112 over the network 110. Examples of external sources 406 include devices in the clinical care environment 10, which are illustrated and described in further detail above with respect to FIGS. 1-3.

The processing device 202 receives and executes instructions from the caregiver guidance system 112 to process the data 402 received over the network 110. In some examples, this includes utilizing artificial intelligence models 404 to process the data 402 and generate guidance system outputs 412. The artificial intelligence models 404 can be applied to process the data 402 and apply the data 402 to certain applications. For instance, a machine learning algorithm can be trained using data 402 from a large number of subjects. The artificial intelligence includes the artificial intelligence models 404 that are configured to analyze data 402 stored in the server memory 204 (including the caregiver actions data 408 and the patient data 410) and apply the data 402 to certain applications. Examples of applications using artificial intelligence are illustrated and described in further detail with respect to FIG. 5.

The server memory 204 includes one or more memories configured to store the data 402 associated with the information received via the network 110 and evaluate the data 402 to create at least one of the guidance system output 412. The server memory 204 can be of various types, including volatile and nonvolatile, removable, and non-removable, and/or persistent media. In some examples, the server memory 204 is an erasable programmable read only memory (EPROM) or flash memory.

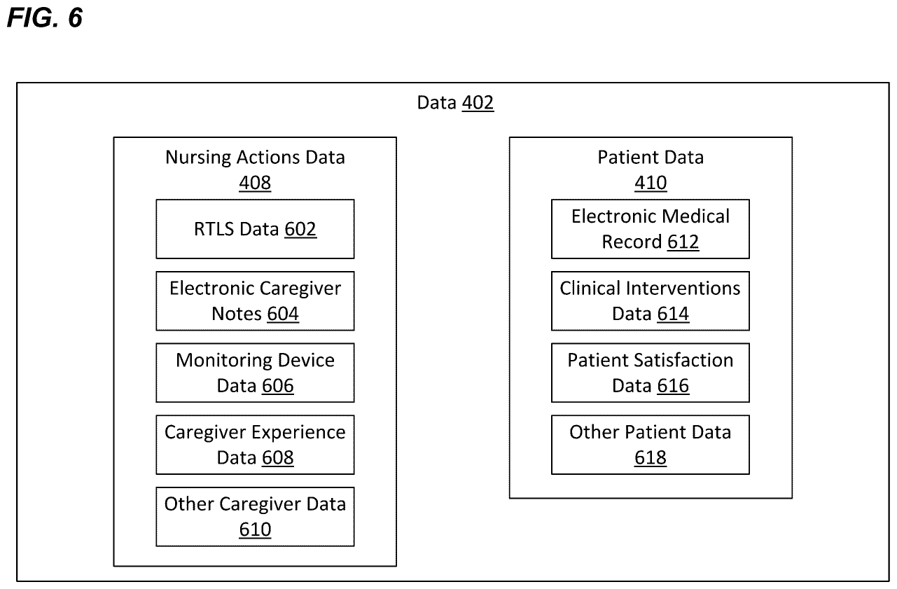

In this example, the server memory 204 stores at least one set of data 402 that includes caregiver actions data 408 and patient data 410. The caregiver actions data 408 includes any action performed by the caregiver that is recorded within the clinical care environment 10. The patient data 410 includes any recorded information that pertains to the patient. Examples of systems and devices for recording the caregiver actions data 408 and patient data 410 are illustrated and described above with respect to FIGS. 1-3. Additional examples of caregiver actions data 408 and patient data 410 are illustrated and described in further detail in FIG. 6.

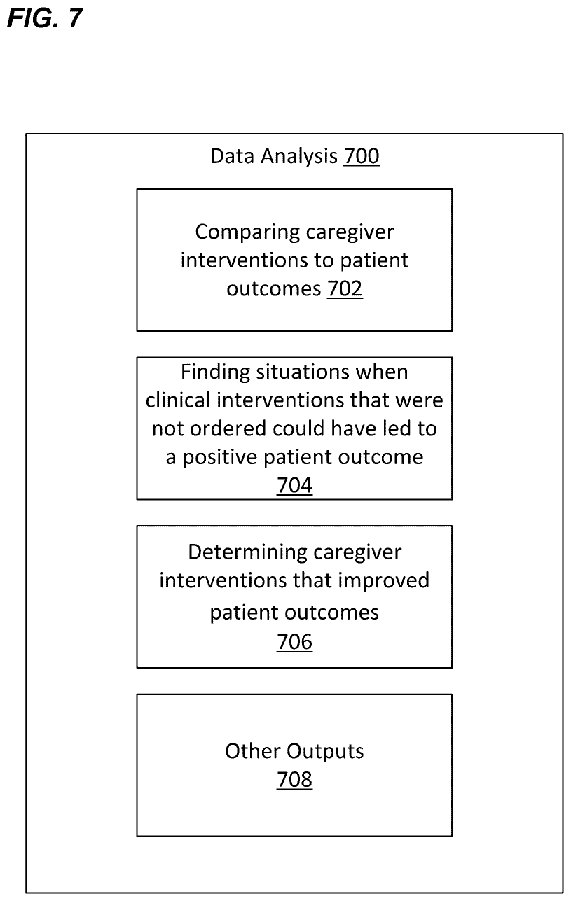

Furthermore, the server memory 204 stores the guidance system outputs 412 that are generated by the processing device 202 after the processing device 202 processes the data 402. In some examples, the processing device 202 utilizes artificial intelligence models 404 to generate the guidance system outputs 412. Examples of guidance system outputs 412 are illustrated and described in further detail with respect to FIGS. 7-14.

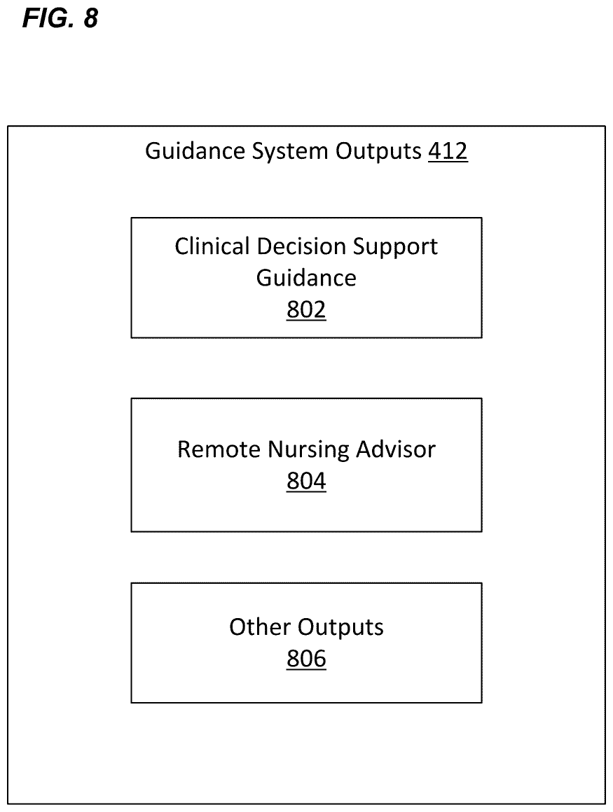

The communications device 106 is substantially the same as discussed above in FIG. 2. The communications device 106 receives processed information, such as the guidance system outputs 412, from the caregiver guidance system 112 and displays the outputs for a caregiver C to review. Examples of communications devices 106 are illustrated and described in further detail with respect to FIGS. 8-14.

FIG. 5 schematically illustrates an example of a method 500 of generating clinical decision support guidance that can be performed by the caregiver guidance system 112. The clinical decision support guidance can include one or more guidance system outputs 412 for display to a caregiver C. As shown in FIG. 5, the method 500 includes step 502 of receiving the caregiver actions data 408 and patient data 410. The caregiver actions data 408 and patient data 410 are illustrated and described in further detail with respect to FIG. 6.

The method 500 includes a step 504 of analyzing the data 402, including the caregiver actions data 408 and the patient data 410. In some examples, the caregiver guidance system 112 analyzes the data 402 by executing instructions stored on the server memory 204. In some examples, the caregiver guidance system 112 analyzes the data by executing instructions stored on the server memory 204 that include an algorithm. In some examples, the instructions include using the artificial intelligence models 404. The step 504 of analyzing the data includes certain examples of data analysis 700, which are described in further detail in FIG. 7.

In step 506, the caregiver guidance system 112 produces guidance system outputs 412 that guide a caregiver C, and specifically an inexperienced caregiver C1, by providing an alert or recommendation. Examples of guidance system outputs 412 are illustrated and described in further detail with respect to FIG. 8.

FIG. 6 schematically illustrates an example of data 402 including caregiver actions data 408 and the patient data 410 that can be used by the caregiver guidance system 112 to generate the guidance system outputs 412. As shown in FIG. 6, the caregiver actions data 408 includes RTLS data 602, electronic caregiver notes 604, monitoring device data 606, caregiver experience data 608, and other caregiver data 610.

The RTLS data 602 is obtained from the RTLS 116. The RTLS data 602 can be used to track the movements of the caregivers C. For example, the RTLS data 602 can include nursing movement patterns and time spent with patients P. In certain examples, the RTLS data 602 includes information specific to how time was spent with each patient such as time spent proactively with a patient to prevent a patient event or time spent as a reaction to a patient event.

Electronic caregiver notes 604 include notes prepared by a caregiver C that describe a patient's condition and clinical interventions that are provided to a patient P. electronic caregiver notes 604 include notes that are inputted into a patient's EMR 612. In some examples, the electronic caregiver notes 604 include descriptions of a patient's visit to a healthcare facility, including the date of the visit, qualitative and quantitative measurements of the patient's condition, the caregivers C that attended to the patient P, and any clinical interventions that were recommended or performed to improve the patient's condition.

Monitoring device data 606 includes data collected by the monitoring device 104. In some examples, the monitoring device 104 collects patient vital signs. The monitoring device 104 is illustrated and described in further detail with respect to FIG. 1. Examples of guidance system outputs 412 that are affected by monitoring device data 606 are illustrated in FIGS. 8-14.

Caregiver experience data 608 includes demographic information about each caregiver C in the clinical care environment 10. The caregiver experience data 608 can include a caregiver's name, age, sex, years of experience, seniority, level of education, nurse healthcare records (including immunization records), and other staffing records.

Other caregiver data 610 includes information that is specific to each healthcare facility 611. Furthermore, other caregiver data 610 can include patient feedback 613 that is provided for each caregiver that relates to the patient's experience with each caregiver C. Further yet, the other caregiver data 610 includes communications between caregivers 615 to discuss patient healthcare or request advice between caregivers C. In certain examples, communications between caregivers 615 can take place via secured messages within a communication system provided by a healthcare facility.

The patient data 410 includes the EMR 612, the clinical interventions data 614, the patient satisfaction data 616, and the other patient data 618.

The EMR 612 (also known as electronic health records or patient health records) includes information related to a patient's medical history. The EMR 612 can be maintained by the healthcare facility over time, and can include clinical data that is relevant to the care that is provided to the patient P. Furthermore, the EMR 612 often include patient demographics such as patient name, age, sex, permanent address, and the like.

The clinical interventions data 614 include a summary of each time a patient P received a clinical intervention from a caregiver C. In certain examples, information pertaining to a clinical intervention will include the date the clinical intervention was administered to the patient P, the reason(s) for administering the clinical intervention (including any symptoms the patient P exhibited), a description of the clinical intervention, and a patient outcome that describes the patient's health after the clinical intervention was administered. In some examples, the clinical interventions data 614 can be included as part of the EMR 612.

Patient satisfaction data 616 includes information provided by the patient P to describe their experience within a healthcare facility or with specific caregivers C. The patient satisfaction data 616 can include feedback on clinical interventions that were administered, their experience with specific caregivers C, and their opinions relating to the healthcare facility.

Other patient data 618 includes patient data 410 that is specific to each healthcare facility. In certain examples, the other patient data 618 includes patient records relating to clinical interventions that were provided at a specific healthcare facility. In certain examples, the other patient data includes data generated from medical equipment that is located at a specific healthcare facility. In certain examples, other patient data 618 includes information relating to operational methods, protocols, or rules that are specific to a healthcare facility.

FIG. 7 schematically illustrates examples of data analysis 700 that can be performed in the method 500 for generating the guidance system outputs 412. As shown in FIG. 7, the data analysis 700 can include comparing caregiver interventions to patient outcomes 702. In some examples, the patient outcomes can be determined from the EMR 612. The caregiver guidance system 112 processes the data 402 to evaluate the effectiveness of certain caregiver interventions that were provided to patients. Caregiver interventions that were more successful in addressing certain patient events are more likely to be recommended to caregivers when a similar patient event occurs. In some examples, the server memory 204 stores large amounts of patient data 410 to strengthen a confidence of its recommendation by analyzing many caregiver interventions and patient outcomes using the processing device 202. Recommendations with greater confidence are more likely to be followed by a first caregiver C1 without requiring further validation from a second caregiver C2 such as an experienced nurse.

In some examples, the data analysis 700 includes isolating situations when clinical interventions that were not ordered by a caregiver C could have led to a positive patient outcome 704. In some examples, the caregiver C has the discretion to order certain clinical interventions to be provided to a patient P while not providing other clinical interventions. In a non-limiting example, a caregiver C may observe that a patient P has returned from an invasive surgery and the surgical site is exhibiting signs of infection. The caregiver C may order the site be cleaned without considering whether the infection has progressed systemically. If the caregiver C did not consider whether the infection has progressed systemically, then the caregiver C might not have ordered a nurse to test a blood sample from the patient P for signs of a systemic infection or provided antibiotics to address the systemic infection. In this example, the data analysis 700 would include analyzing the steps performed to treat the patient (e.g., cleaning the surgical site) while isolating events that could have occurred to prevent the systemic infection (e.g., test the patient's blood and provide antibiotics).

In some examples, the data analysis 700 includes determining caregiver interventions that improved patient outcomes 706. Similar to the above-mentioned example, the caregiver C might have considered a systemic spread of the infection if the surgical site was infected and ordered blood tests be taken and antibiotics be provided to the patient P. If this course of treatment was effective in treating the patient's infection, then this data would be recorded in the patient's EMR 612, and the caregiver guidance system 112 would identify this as a clinical intervention that improved the patient outcome. Therefore, in future situations that are similar to this example, the caregiver guidance system 112 may provide a recommendation to a caregiver C to clean the surgical site, order a blood test, and potentially provide antibiotics if a patient P returns from invasive surgery with the surgical site exhibiting signs of infection. If this situation were to occur in many instances with many positive outcomes, then a confidence rating for the recommendation would increase. If the confidence rating increases above a threshold value, then validation of the clinical intervention from a second nurse C2 might not be required.

In some examples, the data analysis 700 includes other outputs 708. In certain examples, the other outputs 708 include identifying at least one situation when a clinical recommendation should be presented to a first caregiver C1. In certain examples, the caregiver guidance system 112 determines the clinical recommendation should be provided, based on the caregiver actions data 408 or the patient data 410, that the first caregiver C1 is near a patient. In some examples, the caregiver guidance system 112 can determine the first caregiver C1 is near the patient by analyzing the data 402 received from the RTLS 116. In certain examples, the caregiver guidance system 112 determines the clinical recommendation should be provided, based on the caregiver actions data 408 or the patient data 410, that a patient is incorrectly positioned within a patient bed. In certain examples, the caregiver guidance system 112 determines the clinical recommendation should be provided, based on the caregiver actions data 408 or the patient data 410, that a patient's vital signs indicate a patient event is occurring.

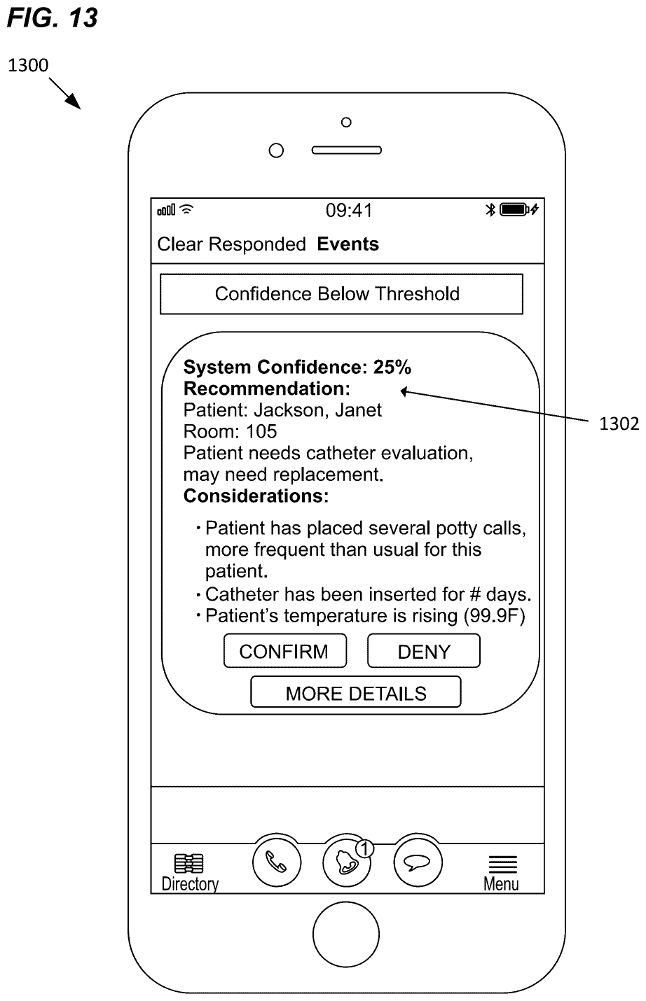

In certain examples, the other outputs 708 include generating a confidence rating for the guidance system outputs 412. As stated above, the confidence rating is dependent upon a number of factors, including a strength of the data provided and other factors discussed above. The strength of the data can be determined by considering at least one of the quantity of experienced, second caregivers C2 that caregiver actions data 408 was collected from, the quantity of actions performed by the experienced, second caregivers C2, and the relevance of the actions performed by the experienced, second caregivers C2 to a current patient event. For example, a confidence rating would be increased if a large quantity of experienced, second caregivers C2 performed a large quantity of actions that were highly relevant to a current patient event. In a non-limiting example, a confidence rating would be increased if a large number of experienced, second caregivers C2 ordered an infection site be cleaned and blood tests be performed on a patient exhibiting signs of infection at a surgical site after an invasive surgery. Furthermore, the confidence rating of a clinical recommendation to perform the same clinical interventions would be increased if a current patient exhibited a similar infection to a surgical site after receiving an invasive surgery. The process of an experienced, second caregiver C2 evaluating a clinical recommendation with a low confidence rating is described and illustrated in further detail with respect to FIG. 13.

In certain examples, the other outputs 708 include a clinical recommendation provided to an inexperienced, first caregiver C1 might require further validation from an experienced, second caregiver C2 if the confidence rating is below a predetermined threshold. In some examples, a clinical recommendation provided to an inexperienced, first caregiver C1 might not require further validation if the confidence rating is above a predetermined threshold.

FIG. 8 schematically illustrates an example of the guidance system outputs 412 that can be generated by the caregiver guidance system 112. As shown in FIG. 8, the guidance system outputs 412 can include clinical decision support guidance 802, a remote nursing advisor 804, and other outputs 806.

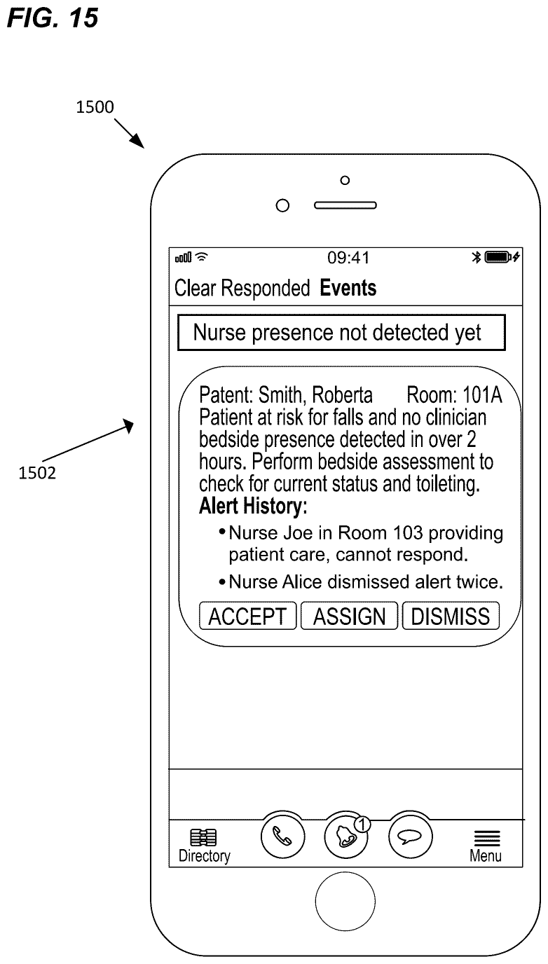

The clinical decision support guidance 802 includes recommendations to guide a caregiver C in providing clinical interventions to a patient P. Specifically, the clinical decision support guidance 802 includes recommendations to guide a first caregiver C1, such as an inexperienced nurse. Methods for producing the clinical decision support guidance 802 are illustrated and described in further detail with respect to FIGS. 5 and 15. Examples of clinical support guidance are illustrated and described in further detail with respect to FIGS. 8-14.

In some examples, the clinical decision support guidance 802 includes a recommendation for a caregiver C to perform a plurality of actions. In some examples, the caregiver guidance system 112 determines a priority each action within the plurality of actions. In a non-limiting example, the caregiver guidance system 112 can recommend certain tasks be performed with one another if they are performed in a similar manner or in a similar location within the healthcare facility.

The remote nursing advisor 804 includes an application to provide a communication channel between a first caregiver C1 and a second caregiver C2 via the caregiver guidance system 112. In some examples, the remote nursing advisor 804 provides a communication channel between an inexperienced caregiver and an experienced caregiver via the caregiver guidance system 112. The remote nursing advisor 804 provides assistance to a first caregiver C1 (e.g., the inexperience caregiver) by allowing the first caregiver C1 to speak with the second caregiver C2 (e.g., the experienced caregiver), such as when the first caregiver C1 has a question about providing clinical interventions to a patient P when a confidence score calculated by the caregiver guidance system 112 is below a predetermined threshold, when the first caregiver C1 lacks experience with the patient event that is occurring, or if the first caregiver is otherwise unfamiliar with the healthcare facility's protocols for resolving a specific patient event. The remote nursing advisor 804 is illustrated and described in further detail in FIGS. 8 and 13.

Other outputs 806 of the guidance system outputs 412 include recommendations for improving the efficiency and safety of caregivers C within the healthcare facility. In certain examples, these recommendations include ideal times to perform rounds (i.e., when to see specific patients P), general advice for managing patients (e.g., providing emotional support), ideal times to check patient vitals or administer medication, efficient ways to manage EMR documentation, recommendations for de-escalating patient emotions, recommendations for disposing of biohazards, and recommendations for efficiently multi-tasking (e.g., performing two tasks that are in close proximity to one another within the healthcare facility).

FIG. 9 illustrates an example of the guidance system outputs 412 that can be generated by the caregiver guidance system 112. The guidance system output 412 can include providing information and a recommendation, requesting feedback from a caregiver, and providing an opportunity to request virtual assistance from an experienced caregiver.

FIG. 9 shows an EMR 612 of a patient P. The EMR 612 receives data from the monitoring device 104 indicating an increase in temperature 902 within the patient's body to thirty-eight degrees centigrade. The caregiver guidance system 112 identifies the increase in temperature 902 to be above a threshold value that indicates the patient P is experiencing a patient event, such as a fever. In response to this identification, the caregiver guidance system 112 can create an alert or recommendation for a caregiver C to view or engage with.

In this example, the caregiver guidance system 112 created a recommendation 904 that identified the patient's temperature was last measured at thirty-eight degrees centigrade, which indicates the patient P is experiencing a patient event. Furthermore, the recommendation includes additional information from the EMR 612 that indicates the patient P is experiencing sepsis. The recommendation 904 identifies that the increase in the patient's temperature may be a symptom of sepsis, and a recommendation is provided to check whether the sepsis is worsening.

In this example, the caregiver guidance system 112 creates a feedback prompt 906 that requests the caregiver C1 to acknowledge whether the recommendation was helpful in improving the patient's condition. The feedback prompt 906 provides an opportunity for caregivers C to provide feedback to the caregiver guidance system 112 when recommendations are provided. In some examples, the feedback from caregivers C is used to train an artificial intelligence models 404.

In this example, the caregiver guidance system 112 creates a remote nursing assistance prompt 908 that provides the opportunity for a caregiver C to request help from a remote nursing advisor 804 as illustrated and described in further detail with respect to FIG. 8.

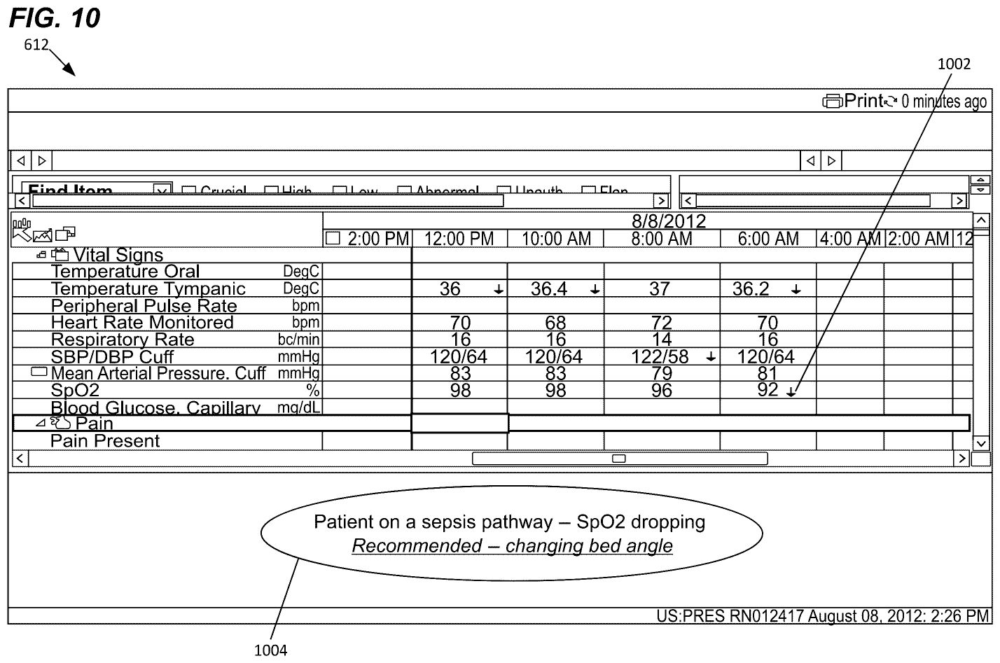

FIG. 10 illustrates another example of the clinical decision support guidance that can be generated by the caregiver guidance system 112. In this example, the guidance system output 412 includes reviewing a patient's EMR 612 and providing a recommendation. The EMR 612 receives data from the monitoring device 104 indicating the patient's blood oxygen saturation decreased to ninety-two percent. The caregiver guidance system 112 identified the patient's blood oxygen saturation to be below a threshold value that indicates the patient P is experiencing a patient event, such as sepsis. In response to this identification, the caregiver guidance system 112 can create an alert or recommendation for a caregiver C to view or engage with.

In this example, the caregiver guidance system 112 creates a sepsis recommendation 1004 that identifies the patient's blood oxygen saturation was last measured at ninety-two percent, which indicates the patient P is experiencing a patient event. Furthermore, the sepsis recommendation 1004 includes additional information from the EMR 612 that indicates the patient P is experiencing sepsis. The recommendation 904 identifies that the decrease in the patient's blood oxygen saturation may be a symptom of sepsis, and a recommendation is provided to the caregiver C to change the bed angle of the patient P to improve oxygenation and respiratory mechanics.

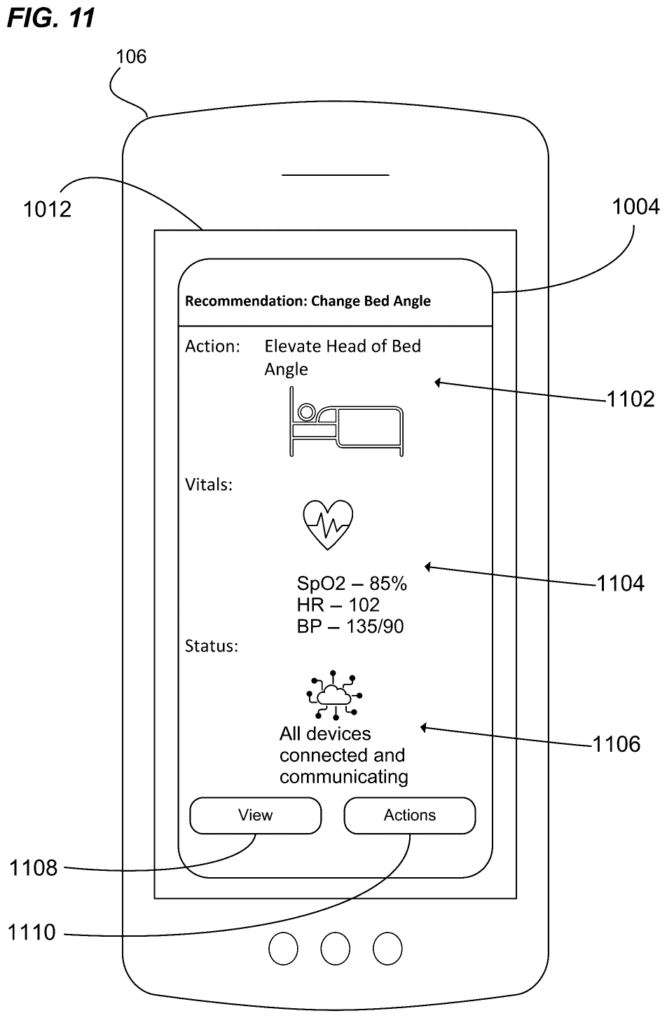

FIG. 11 illustrates an example of a communications device 106 displaying an example of clinical decision support guidance that can be generated by the caregiver guidance system 112. In this example, the communications device 106 is a smartphone that displays the clinical recommendations via a smartphone application.

The communications device 106 displays additional information relating to the sepsis recommendation 1004, including an action 1102, vital signs of the patient 1104, and a connectivity status 1106 of the communications device 106 with other devices connected via the network 110. In this example, the action 1102 recommends the caregiver C to elevate the patient's head to improve oxygenation in response to the patient's low blood oxygen saturation. In this example, the vital signs of the patient 1104 indicate the patient's blood oxygen saturation has further decreased to eighty-five percent while also displaying a patient heart rate and blood pressure (including a systolic and diastolic blood pressure). In this example, the connectivity status 1106 indicates all devices are connected to the network 110 and communicating properly.

The display module also displays icons on the bottom of the screen, including a view icon 1108 and an actions icon 1110. The view icon 1108 allows the caregiver C to view additional information that is provided with the sepsis recommendation 1004. The actions icon 1110 allows the caregiver to document actions that were taken in response to the recommendation or request input from a second caregiver C2, such as an experienced nurse.

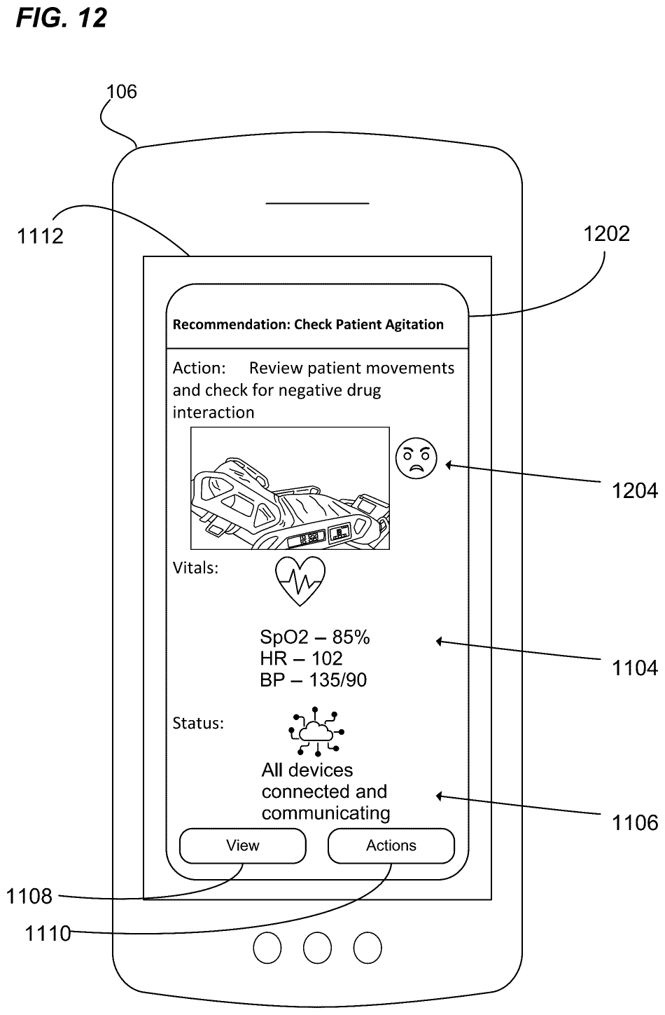

FIG. 12 illustrates an example of a communications device 106 displaying another example of a clinical recommendation that can be generated by the caregiver guidance system 112. In this example, the communications device 106 displays the vital signs of the patient 1104, the connectivity status 1106, the view icon 1108, and the actions icon 1110 of FIG. 11. Additionally, the communications device 106 displays another example of a clinical recommendation including a patient agitation recommendation 1202. The patient agitation recommendation 1202 includes an action 1204 that recommends the caregiver C review patient movements and check for negative drug interactions with the patient P. In some examples, this recommendation can be generated by the caregiver guidance system 112 based on an analysis of data 402 received from the RTLS 116, the monitoring device 104, or other camera-based systems that record patient movement.

FIG. 13 schematically illustrates an example of an alert 1300 received when a confidence rating produced by the caregiver guidance system 112 is below a predetermined threshold. The alert 1300 notifies a caregiver C that the confidence rating generated by the caregiver guidance system 112 is below a predetermined threshold (e.g., twenty-five percent) 1302. The alert 1300 includes a recommendation to provide a clinical intervention to a patient, such as evaluate a catheter for replacement, and a plurality of considerations to provide context for why the recommendation was provided. Furthermore, the alert 1300 includes options to confirm the accuracy of the recommendation, deny the accuracy of the recommendation, or view additional details. In some examples, the first alert is sent to a first caregiver C1 that is an inexperienced nurse.

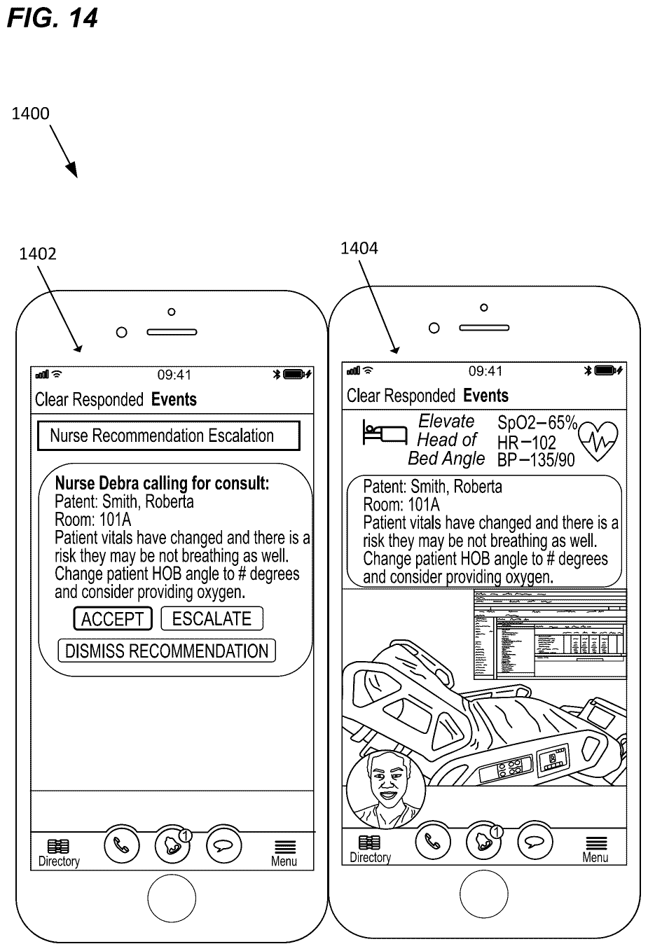

FIG. 14 schematically illustrates an example of an alert 1400 received when a clinical recommendation produced by the caregiver guidance system 112 is escalated for further review by an experienced caregiver. The alert 1400 notifies a caregiver C that a recommendation 1402 has been escalated to a second caregiver C2, such as an experienced nurse. The alert 1400 includes a description of the first caregiver C1 that escalated the recommendation to a second nurse. Furthermore, the alert 1400 includes a description of the patient P, a location of the patient, and any electronic caregiver notes 604 prepared by the first caregiver C1. The second caregiver C2 may select icons that allow the second caregiver C2 to accept a call from the first caregiver C1, escalate the recommendation to an additional caregiver C, or dismiss the recommendation 1402 if the second caregiver C2 deems the recommendation 1402 is inaccurate.

The alert 1400 also includes patient information 1404 relating to the recommendation 1402, such as patient vital signs, the EMR 612, and any electronic caregiver notes 604 prepared for the patient. In some examples, the alert 1400 is provided to the second caregiver C2 with the other alerts described and illustrated in further detail with respect to FIGS. 12-13 when the first caregiver C1 escalates the recommendation received in the alert 1300 of FIG. 13.

FIG. 15 schematically illustrates an example of an alert 1500 received when an inexperienced caregiver does not follow a clinical recommendation produced by the caregiver guidance system 112. The alert 1500 includes a notification 1502 to a caregiver C if another caregiver C does not follow the recommendation that was provided by the caregiver guidance system 112. The notification 1502 includes a description of the patient P, electronic caregiver notes 604, and a history of alerts from the caregiver guidance system 112 or from other caregivers C. In some examples, the alert 1500 is received by a second caregiver C2 (an experienced nurse) when the first caregiver C1 (an inexperienced nurse) fails to follow the recommendation. It can be desirable for the second caregiver C2 to receive an alert when the recommendation is not followed because it allows the second caregiver C2 to identify instances where the first caregiver C1 may provide a suboptimal clinical intervention.

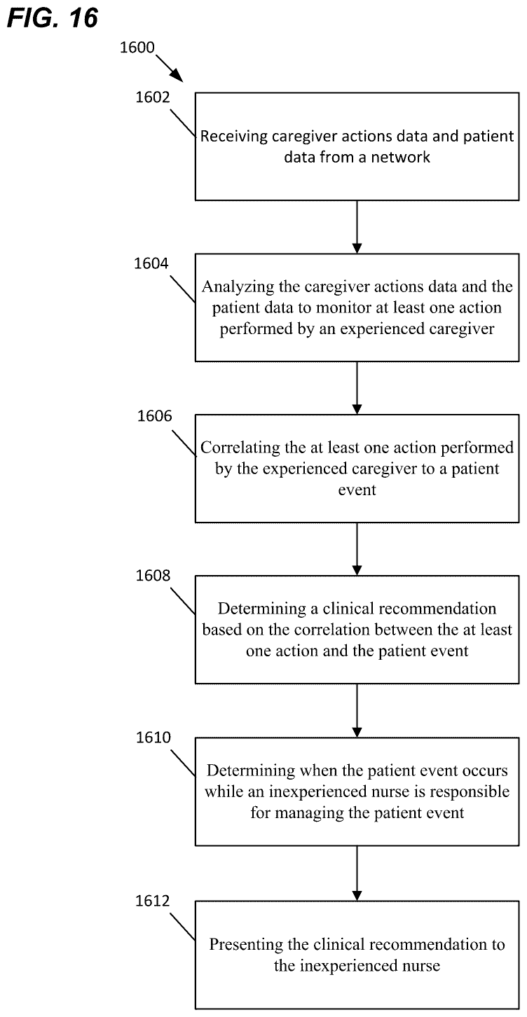

FIG. 16 schematically illustrates an example of a method 1600 of determining a clinical recommendation that can be performed by the caregiver guidance system 112. As shown in FIG. 16, the method 1600 includes a step 1602 of receiving over the network 110 caregiver actions data 408 and patient data 410. The caregiver actions data 408 and patient data 410 are illustrated and described in further detail with respect to FIG. 6. Transmission of the data 402 to the caregiver guidance system 112 over the network 110 is illustrated and described in further detail with respect to FIGS. 1-2.

The method 1600 includes a step 1604 of analyzing the caregiver actions data 408 and patient data 410 to monitor at least one action performed by an experienced caregiver. In certain examples, the experienced caregiver is a second caregiver C2 as discussed above. The actions of the experienced caregiver are monitored through a plurality of different systems, including the RTLS 116, the monitoring device 104, other camera-based systems, other caregivers C, and other sources. Information from these sources is received by the caregiver guidance system 112 over the network 110 and analyzed.

The method 1600 includes a step 1606 of correlating the at least one action performed by the experienced caregiver to a patient event. In certain examples, the at least one action performed by the experienced caregiver is a clinical intervention. In certain examples, the patient event is a medical condition that is treated by the clinical intervention from a caregiver C. Examples of actions performed by caregivers C to aid a patient P that is experiencing a patient event are illustrated and described in further detail with respect to FIGS. 9-15.

The method 1600 includes a step 1608 of determining a clinical recommendation based on the correlation between the at least one action and the patient event. In some examples, the clinical recommendation is determined by comparing the caregiver interventions to patient outcomes in an EMR record, as illustrated and described in further detail with respect to FIG. 5. Furthermore, in some examples, the clinical recommendation is determined by identifying instances when clinical therapeutic interventions that were not ordered by a caregiver might have led to a more positive patient outcome, as illustrated, and described in further detail with respect to FIG. 5. Further yet, in some examples, if the caregiver guidance system 112 may connect an experienced caregiver with an inexperienced caregiver via the caregiver guidance system 112 if the caregiver guidance system 112 is unable to identify relevant patient cases that are similar to the current patient event, if the caregiver guidance system 112 determines the patient's health is deteriorating, or if the inexperienced caregiver is completing nursing actions that do not follow the clinical recommendation.

The method 1600 can include a step 1610 of determining when the patient event occurs while an inexperienced nurse is responsible for managing the patient event. The caregiver guidance system 112 determines when a patient event is occurring by analyzing the data 402 received over the network 110. In certain examples, the caregiver guidance system 112 may determine a patient event is occurring by reviewing the vital signs of a patient P that are being recorded by the monitoring device 104 and transmitted to the caregiver guidance system 112 via the network 110. Furthermore, the caregiver guidance system 112 may determine when an inexperienced nurse is responsible for managing the patient event by reviewing caregiver staffing records for the healthcare facility and caregiver demographics. The caregiver staffing records, and the caregiver demographics are illustrated and described in further detail in FIG. 6.

The method 1600 can include step 1612 of presenting the clinical recommendation to the inexperienced nurse. In certain examples, the clinical recommendation is presented to the inexperienced nurse by transmitting the clinical recommendation from the caregiver guidance system 112 over the network 110 to a communications device 106, as illustrated and described in further detail with respect to FIG. 2. In some examples, the communications device 106 includes a computer workstation as shown in the examples of FIGS. 8 and 9, or a mobile device such as a smartphone having a touch display as illustrated and described in FIGS. 10-14.

The various examples described above are provided by way of illustration only and should not be construed to be limiting in any way. Various modifications can be made to the examples described above without departing from the true spirit and scope of the disclosure.

What is claimed is:

1. A caregiver guidance system for generating at least one clinical recommendation, the system comprising:
at least one processing device; and
a memory device storing instructions which, when executed by the at least one processing device, cause the at least one processing device to:
receive caregiver actions data associated with an experienced caregiver and patient data associated with a patient;
analyze the caregiver actions data to identify at least one action performed by the experienced caregiver in response to a patient event detected from the patient data;
correlate the at least one action performed by the experienced caregiver to the patient event;
determine a clinical recommendation based on the correlation between the at least one action and the patient event;
determine when the patient event occurs while an inexperienced caregiver is managing the patient event; and
present the clinical recommendation to the inexperienced caregiver.

2. The caregiver guidance system of claim 1, wherein the instructions, when executed by the at least one processing device, further cause the at least one processing device to:
receive the caregiver actions data and the patient data from a camera-based observation system configured to record video of the at least one action with at least one patient.

3. The caregiver guidance system of claim 1, wherein the instructions, when executed by the at least one processing device, further cause the at least one processing device to:
receive the caregiver actions data and the patient data from a real time location system configured to record the caregiver actions data by monitoring a location of the caregiver.

4. The caregiver guidance system of claim 1, wherein the instructions, when executed by the at least one processing device, further cause the at least one processing device to:
receive the caregiver actions data and the patient data from at least one of a patient bed, an electronic medical record, or a patient vital sign detector.

5. The caregiver guidance system of claim 1, wherein the instructions, when executed by the at least one processing device, further cause the at least one processing device to:
identify at least one situation to present the clinical recommendation to the inexperienced caregiver, wherein the at least one situation includes when the inexperienced caregiver is near a patient or when the patient event is occurring.

6. The caregiver guidance system of claim 1, wherein the instructions, when executed by the at least one processing device, further cause the at least one processing device to:
analyze the caregiver actions data corresponding to a plurality of experienced caregivers;
determine a most common action performed by the plurality of experienced caregivers in response to the patient event; and
provide the most common action as the clinical recommendation.

7. The caregiver guidance system of claim 6, wherein the instructions, when executed by the at least one processing device, further cause the at least one processing device to:
determine a strength of the caregiver actions data;
generate a confidence rating based on the strength of the caregiver actions data; and
issue an alert when the confidence rating is less than a confidence threshold value.

8. The caregiver guidance system of claim 7, wherein the instructions, when executed by the at least one processing device, further cause the at least one processing device to:
request validation from a second caregiver when the confidence rating is less than the confidence threshold value.

9. The caregiver guidance system of claim 1, wherein the instructions, when executed by the at least one processing device, further cause the at least one processing device to:
monitor actions by the inexperienced caregiver;
determine whether the actions by the inexperienced caregiver conform to the clinical recommendation; and
issue an alert when the actions by the inexperienced caregiver do not conform to the clinical recommendation.

10. The caregiver guidance system of claim 9, wherein the instructions, when executed by the at least one processing device, further cause the at least one processing device to:
request validation from an experienced caregiver when the actions by the inexperienced caregiver do not conform to the clinical recommendation.

11. A method for determining a clinical recommendation comprising:
receiving caregiver actions data associated with an experienced caregiver and patient data associated with a patient;
analyzing the caregiver actions data to determine at least one action performed by an experienced caregiver in response to a patient event detected from the patient data;
correlating the at least one action to the patient event;
determining a clinical recommendation based on the correlation between the at least one action and the patient event;
determining when the patient event occurs when an inexperienced caregiver is managing the patient event; and
presenting the clinical recommendation to the inexperienced caregiver.

12. The method for determining a clinical recommendation of claim 11, wherein the caregiver actions data and patient data are received from at least one of a camera-based observation system, a real time location system, a patient bed, an electronic medical record system, and a patient vital sign detector.

13. The method for determining a clinical recommendation of claim 11, further comprising:
identifying at least one situation to present the clinical recommendation to the inexperienced caregiver, wherein the at least one situation includes when the inexperienced caregiver is near a patient or when the patient event is occurring.

14. The method for determining a clinical recommendation of claim 11, further comprising:
analyzing caregiver actions data corresponding to a plurality of experienced caregivers;
determining a most common action that is performed by the plurality of experienced caregivers in response to the patient event; and
providing the most common action as the clinical recommendation.

15. The method for determining a clinical recommendation of claim 14, further comprising:
determining a strength of the caregiver actions data;
generating a confidence rating based on the strength of the caregiver actions data; and
issuing an alert when the confidence rating is less than a confidence threshold value.

16. The method for determining a clinical recommendation of claim 15, further comprising:
requesting validation from an experienced caregiver when actions by the inexperienced caregiver do not conform to the clinical recommendation.

17. A communications device for displaying at least one clinical recommendation, the communications device comprising:
at least one processing device; and
a memory device storing instructions which, when executed by the at least one processing device, cause the at least one processing device to:
detect a patient event;
determine a level of experience of a caregiver proximate the patient event;
receive at least one clinical recommendation when the level of experience of the caregiver is below a predetermined threshold;
present the at least one clinical recommendation to the caregiver.

18. The communications device of claim 17, wherein the instructions, when executed by the at least one processing device, further cause the at least one processing device to:
identify at least one situation to present the clinical recommendation to the caregiver, wherein the at least one situation includes when the caregiver is near a patient or when the patient event is occurring.

19. The communications device of claim 17, wherein the at least one clinical recommendation is presented on at least one of a bed and a vital signs spot monitor.

20. The communications device of claim 17, wherein the instructions, when executed by the at least one processing device, further cause the at least one processing device to:
analyze caregiver actions data corresponding to a plurality of experienced caregivers;
determine a most common action that is performed by the plurality of experienced caregivers in response to the patient event; and
provide the most common action as the clinical recommendation.
